(19) United States
(12) Patent Application Publication
(10) Pub. No.: US 2013/0274214 A1
(43) Pub. Date: Oct. 17, 2013

(54) METHOD FOR TREATING BLEPHARITIS

(71) Applicant: Kurt E. Brubaker, Shady Cove, OR (US)

(72) Inventor: Kurt E. Brubaker, Shady Cove, OR (US)

(73) Assignee: INSPIRE PHARMACEUTICALS, INC., Whitehouse Station, NJ (US)

(21) Appl. No.: 13/976,725

(22) PCT Filed: Dec. 28, 2011
(86) PCT No.: PCT/US2011/067599

§ 371 Date: Jun. 27, 2013

Related U.S. Application Data
(60) Provisional application No. 61/427,962, filed on Dec. 29, 2010.

(51) Int. Cl. A61K 31/7052; A61K 31/23

(52) U.S. Cl. CPC A61K 31/7052; A61K 31/23
USPC 514/29

(57) ABSTRACT

This invention relates to methods of treating blepharitis. The methods comprise identifying a patient suffering from blepharitis, and topically administering to the eyes of the patient an effective amount of azithromycin and an effective amount of a retinoid. The method is useful in relieving blepharitis signs and symptoms. This invention also relates to a pharmaceutical composition comprising azithromycin and a retinoid such as retinyl palmitate.

### TECHNICAL FIELD

This invention relates to methods of treating blepharitis by administering azithromycin and a retinoid to a subject. The method is useful in relieving blepharitis signs and symptoms. This invention also relates to a pharmaceutical composition comprising azithromycin and a retinoid such as retinyl palmitate.

### BACKGROUND OF THE INVENTION

Blepharitis is a chronic disorder producing inflammation of the anterior and posterior lid margin, with involvement of skin and its related structures (hairs and sebaceous glands), the mucocutaneous junction, and the meibomian glands. It can also affect the conjunctiva, tear film, and the corneal surface in advanced stages. Blepharitis is commonly classified into anterior or posterior blepharitis, with anterior affecting the lash bearing region of the lids, and posterior primarily affecting the meibomian gland orifices (American American Academy of Ophthalmology, Blepharitis. 2003; Thygeson, Arch Ophthalmol., 1946, 36:938-942; Foulks, Ocul Surf 2003;1(3):107-120). Blepharitis is one of the most common ocular disorders seen by ophthalmologists and has no cure to date or FDA-approved treatments for this condition.

Blepharitis in its mild form is usually undiagnosed and rarely managed. In one study, the prevalence of blepharitis was estimated at 10% in the general population (Claoué, Eye, 1997, 11(6):865-868) but is probably higher in the elderly. Blepharitis is associated with a broad spectrum of ocular symptoms ranging from mild transient irritation to persistent irritation, burning, itching, redness, pain, ocular fatigue and vision disturbances.

Blepharitic changes limited primarily to the posterior lid margin arise predominantly from pathological processes centered around the meibomian glands. The meibomian glands are holocrine glands that supply the lipids, which form the external oily layer of the precorneal tear film. It is the alteration in this excretory process and the composition of tear film lipids that cause the clinical manifestations seen with this disease.

Clinical and laboratory investigations in recent years have identified several forms of meibomian gland disease/dysfunction (MGD). McCulley et al. (Ophthalmology, 1982, 89: 1173-1180) have described three forms of meibomitis characterized by biomicroscopic changes in the meibomian glands and ducts. “Secondary meibomitis” represents a localized inflammatory response in which the meibomian glands are secondarily inflamed in a spotty fashion from an anterior lid margin blepharitis. Both “meibomian seborrhea” and “primary meibomitis” produce generalized gland dysfunction, but differ with regard to the underlying glandular abnormality. Meibomian seborrhea is characterized by excessive meibomian secretion in the absence of inflammation (hypersecretory form). Primary meibomitis, by contrast, is distinguished by stagnant and inspissated meibomian secretions (obstructive form).

Acne rosacea, seborrheic dermatitis, psoriasis, atopy and hypersensitivity to bacterial products may all contribute to the etiology of blepharitis (Cher, Mod Med Austr., 1997, 52-62). It is generally assumed that infection plays a role in anterior blepharitis (Thygeson, 1946; Dougherty, 1984; Smith, CLAO J., 1995, 21(3):200-207), and cell-mediated immune responses to staphylococcal antigens has been emphasized (Ficker, Am J Ophthalmol., 1991, 15; 111(4):473-479).

It has been suggested that chronic blepharitis can have an inflammatory etiology that is not associated with infection (Seal, Br J Ophthalmol., 1985, 69(8):604-611). Some studies have demonstrated that only a small proportion of patients with meibomian gland dysfunction (Mathers, Cornea., 1996,15(2):110-119) and blepharitis have evidence of an active infection or show the production of staphylococcal toxins (Seal, Ophthalmology., 1990, 97(12):1684-1688). Histological studies have detected inflammatory cell infiltrates containing neutrophils and lymphocytes in the corium and epidermis of blepharitis patients. A chronic nongranulomatous inflammatory reaction is observed in most cases of chronic blepharitis and blepharoconjunctivitis (Yanoff, Ocular pathology. 3rd ed. Lippincott Williams & Wilkins Publishers, 1989;171-172). The pathophysiology of blepharitis is not well understood, but current consensus is that bacteria, altered meibum lipid composition and inflammation are the major contributors to the process.

The inflammatory aspects of blepharitis have been treated with topical steroids as well as systemic tetracycline for three months or longer. However, the well known side effects of steroid use and long term systemic antibiotic use make these treatment regimes less than optimal. Further, antibiotic ointments have been used to treat the overgrowth of normal bacterial flora in this disease. However, topical antibiotic treatment has not been used to address the inflammatory aspects of blepharitis.

Keratin is a tough, fibrous protein that is not water-soluble and is the main component of hair, nails and the epidermis (Ong, et al., Current Eye Research, 10, 1113-1119 (1991)). Keratinization is a process which involves the conversion of epithelial or other cell types in to cells that are largely comprised of keratin (Tseng, et al., Ophthalmology, 91, 545-552 (1984), and Jester, et al., Inv. Ophthal. Vis. Sci. 30, 927-935 (1989). Keratinization of cells in ocular surface tissues such as the conjunctiva, goblet cells and the meibomian glands may play a role in the development of blepharitis (Nicholaides, et al., Inv. Ophthal. Vis. Sci. 30, 946-951 (1989). The secretions from the squamous epithelial cells and goblet cells in the conjunctiva contribute to the tear film mucin and aqueous components. Overexpression of keratin, or hyperkeratinization, of these cell types may impact both the quality and quantity of the tear film. Keratinization of the meibomian glands may occur on the lid margin, obstructing the orifice of the glands, or inside the glands to the epithelial lining of the glands. Hyperkeratinization either in the glands or of the surface epithelium may alter the lipid secretions which comprise the outer layer of the tear film.

Retinoids include Vitamin A (retinol), retinoic acid, and retinyl palmitate as well as related compounds that are synthetic or naturally occurring cellular components or metabolites. The effects of RA and synthetic derivatives are mediated by two classes of nuclear receptors, the retinoic acid receptors which belong to the erbA-related steroid/thyroid nuclear receptor superfamily and the retinoid X receptors which also belong to the same super family of steroid/thyroid hormones (Gorodeski, et al., Am. J. Physiol. Cell. Physiol. 275, 758-765 (1998).

Vitamin A and related retinoids are involved in the maintenance of mucosal membranes via control of the proliferation and differentiation of epithelial cells. A deficiency of retinoids results in a gradual change of the ocular mucosa to a non-secretory keratinized epithelium. (Kobayashi, et al., Ophthalmologica, 211, 358-361 (1997)). Retinoic acid plays a fundamental role in cell proliferation, and cell differentiation and it may also prevent malignant transformation (Darmon, 1991, Sem. Dev. Biol. 2:219).

Retinoids have been utilized to treat a number of conditions involving keratinization of epithelial tissue, including: acne vulgaris, psoriasis, wound healing and premalignant lesions (Kligman, A., Cutis, 39, 486-488 (1987). Formulations containing retinoids have also been utilized to treat ocular disorders involving the epithelium, such as dry eye, Stevens-Johnson syndrome (Kobayashi, et al., Ophthalmologica, 211, 358-361 (1997); Selek, et al., Eur. J. Ophthalmol, 10, 121-127 (2000) and Kim, et al., Amer. J. Oph, 147, 206-213.e3 (2009)). Topical retinoid formulations include ointments and liquid formulations that may be applied 2-4 times per day for one or more months. Increases in goblet cell density in ocular mucosal tissue, tear break up time and Schirmer score measurements have been noted following topical retinoid therapy.

Systemic treatment with retinoids for epithelial keratinization disorders may be effective, but side effects due to toxicity are common (Kligman, A., Cutis, 39, 486-488 (1987). Topical preparations allow for sufficient efficacy at the site of the disorder, yet avoid systemic side effects. Side effects of topical retinoid formulations include irritation and redness during the initial period of therapy. Practices to reduce the side effects of topical retinoid formulations include a reduction in the concentration of the active ingredient, a reduction in frequency of administration or discontinuation of therapy, which make the retinoid treatment less effective.

Azithromycin is a macrolide antibiotic. AZASITE® (azithromycin ophthalmic solution) is a 1% sterile aqueous topical ophthalmic solution of azithromycin formulated in DURASITE® (polycarbophil, edetate disodium, sodium chloride). AZASITE® is approved by the U.S. Food and Drug Administration (FDA) for treatment of bacterial conjunctivitis, caused by susceptible isolates of CDC coryneform group G, Haemophilus influenzae, Staphylococcus aureus, Streptococcus mitis group, and Streptococcus pneumoniae (AZASITE® Package Insert, 2007). The recommended dosage regimen for the treatment of bacterial conjunctivitis is as follows: instill 1 drop in the affected eye(s) twice daily, 8 to 12 hours apart for the first 2 days and then instill 1 drop in the affected eye(s) once daily for the next 5 days (AZASITE® Package Insert, 2007).

Azithromycin has immunomodulatory and anti-inflammatory effects that are separate from the antimicrobial effects. Studies have been conducted that demonstrate a reduction in inflammatory cell influx, pro-inflammatory mediator release (e.g, cytokines and chemokines), and tissue remodeling mediators such as matrix metalloproteinases (Amsden, G. W., Journal of Antimicrobial Chemotherapy, 55, 10-21 (2005), Li, D. Q., Invest Ophthalmol Vis Sci, 51(11), 5623-9 (2010)).

Topical application of azithromycin to the ocular surface has been shown to improve the quality of the meibomian gland secretions. Daily administration of topical azithromycin in subjects with meibomian gland dysfunction improved the physicochemical properties of the meibum towards that of normal subjects (Foulks, G. N., Cornea, 29(7), 781-8 (2010)).

Despite the high prevalence of the blepharitis, present therapies often result in poor patient compliance and disappointing results. Therefore, there is a need for an effective and safe method to treat blepharitis.

### SUMMARY OF THE INVENTION

The present invention is directed to a method for treating blepharitis. The method comprises the steps of: identifying a patient suffering from blepharitis, and topically administering to the patient an effective amount of azithromycin or a pharmaceutically acceptable salt thereof and an effective amount of a retinoid. Azithromycin and the retinoid can be administered either sequentially or by co-administration. Preferred azithromycin is azithromycin monohydrate and preferred retinoid is retinyl palmitate. An effective concentration of azithromycin is about 0.5%-2% (w/v), and an effective concentration of retinyl palmitate is about 0.01-0.1% (w/v). An effect dosing regimen would consist of topical administration of 1-2 drops (approximately 50 microliters) in each eye once or twice a day.

The present invention is also directed to a pharmaceutical composition comprising active ingredients and a pharmaceutically acceptable carrier, wherein the active ingredients consisting essentially of 0.5-2% (w/v) of azithromycin and 0.01-1% (w/v) retinyl palmitate. The pharmaceutical composition preferably is an ophthalmic solution or suspension.

### DETAILED DESCRIPTION OF THE INVENTION

The inventor has discovered an effective method for treating blepharitis by administering azithromycin and a retinoid to a patient suffering from blepharitis. The inventor has discovered that combined administration of azithromycin and a retinoid is more effective in treating blepharitis than the single administration of either azithromycin or a retinoid.

The present invention is directed to a method for treating blepharitis. The method comprises the steps of: identifying a patient suffering from blepharitis, and topically administering to the patient an effective amount of azithromycin or a pharmaceutically acceptable salt thereof and an effective amount of a retinoid. Azithromycin and the retinoid can be administered to the patient separately or co-administered in one single formulation.

In another embodiment, the method comprises the steps of: identifying a patient suffering from blepharitis, and administering to the patient a pharmaceutical composition comprising active ingredients and a pharmaceutically acceptable carrier, wherein the active ingredients consisting essentially of an effective amount of azithromycin and an effective amount of a retinoid. The active ingredients only include azithromycin and a retinoid and those that do not materially affect the basic and novel characteristics of the claimed invention.

“Retinoids” as used in this application, refer to a class of compounds consisting of four isoprenoid units joined in a head-to-tail manner. All retinoids may be formally derived from a monocyclic parent compound containing five carbon-carbon double bonds and a functional group at the terminus of the acyclic portion. Retinoids are a class of chemical compounds that are chemically related to vitamin A (retinol). Examples of retinoids useful in the present invention include: vitamin A (retinol), retinyl palmitate, retinal, tretinoin (retinoic acid), isotretinoin, etretinate, acitretin, tazarotene, bexarotene, and adapalene. Retinyl palmitate is a preferred compound for this invention.

“An effective amount” as used herein, is meant an amount that has a therapeutic effect, which reduces the signs and/or symptoms of blepharitis. In the present method, the effective concentration of azithromycin is about 0.5%-2% (w/v), e.g., about 1%, and an effective concentration of retinyl palmitate is about 0.01-0.1% (w/v), e.g., about 0.05%. An effect dosing regimen would consist of topical administration of 1-2 drops (approximately 50 microliters) in each eye once or twice a day. In one embodiment, the effective concentration of azithromycin is 1% (w/v), and an effective amount of retinyl palmitate is 0.05% (w/v).

“About” as used in this application, refers to +10% of the recited value.

### Pharmaceutical Composition

The present invention is directed to a pharmaceutical composition comprising azithromycin or a pharmaceutically acceptable salt thereof, a retinoid, and a pharmaceutically acceptable carrier. Preferred azithromycin is azithromycin monohydrate and preferred retinoid is retinyl palmitate. The pharmaceutical composition preferably is an ophthalmic solution or suspension. A preferred pharmaceutical composition comprises active ingredients and a pharmaceutically acceptable carrier, wherein the active ingredients consisting essentially of 0.5-2% of azithromycin and 0.01-1% retinyl palmitate.

The topical solution containing azithromycin and a retinoid can contain a physiologically compatible vehicle, as those skilled in the ophthalmic art can select using conventional criteria. The ophthalmic vehicles include, but are not limited to, saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

Preferred ophthalmic formulations of azithromycin and retinoids suitable for the present method are those disclosed in U.S. Pat. Nos. 6,239,113, 6,569,443 and 7,056,893; the formulations of which are incorporated herein by reference. For example, the formulation is an aqueous polymeric suspension comprising water, azithromycin, retinyl palmitate, and 0.1 to 10% of a polymeric suspending agent. The polymeric suspending agent comprises a water-swellable water-insoluble crosslinked carboxy-vinyl polymer. For example, the polymeric suspending agent comprises least 90% (w/v) acrylic acid monomers and 0.1% to 5% (w/v) crosslinking agent. The formulations may further comprise polyvinyl alcohols and providone.

AZASITE® (azithromycin ophthalmic solution), which is a 1% (w/v) sterile aqueous topical ophthalmic solution of azithromycin formulated in DURASITE® (polycarbophil, edetate disodium, sodium chloride), is a preferred ophthalmic formulation. Retinyl palmitate can be added to AZASITE® to form a combination formulation. The preferred ophthalmic formulations are able to keep prolonged high azithromycin and retinyl palmitate concentrations on the ocular surface, thus facilitating its penetration into the eye tissues.

The formulation optionally includes a preservative, such as benzalkonium chloride and other inactive ingredients such as EDTA. For a short term use of less than two weeks, preferably less than one week, benzalkonium chloride has the benefit of increasing the penetration of azithromycin into eye tissues. However, for chronic (over two weeks) use, preferred formulations are those without any preservatives due to the potential for damage to the corneal epithelium that may result from long term, frequent exposure to preservatives such as benzalkonium chloride. The formulations without preservatives are prepared in a unit dose and stored in a single-use container.

The pH of the formulation is typically adjusted by adding any physiologically and ophthamologically acceptable pH adjusting acids, bases or buffers to within the range of about 5 to 7.5; preferably 6 to 7. Examples of acids include acetic, boric, citric, lactic, phosphoric, hydrochloric, and the like, and examples of bases include sodium hydroxide, sodium phosphate, sodium borate, sodium citrate, sodium acetate, sodium lactate, tromethamine, THAM (trishydroxymethylamino-methane), and the like. Salts and buffers include citrate/dextrose, sodium bicarbonate, ammonium chloride and mixtures of the aforementioned acids and bases.

The osmotic pressure of the aqueous ophthalmic composition is generally from about 200 to about 400 milliosmolar (mOsM), more preferably from 260 to 340 mOsM. The osmotic pressure can be adjusted by using appropriate amounts of physiologically and ophthamologically acceptable ionic or non-ionic agents. Sodium chloride is a preferred ionic agent, and the amount of sodium chloride ranges from about 0.01% to about 1% (w/v), and preferably from about 0.05% to about 0.45% (w/v). Equivalent amounts of one or more salts made up of cations such as potassium, ammonium and the like and anions such as chloride, citrate, ascorbate, borate, phosphate, bicarbonate, sulfate, thiosulfate, bisulfate, sodium bisulfate, ammonium sulfate, and the like can be used in addition to or instead of sodium chloride to achieve osmolality within the above-stated range. Further, non-ionic agents such as mannitol, dextrose, sorbitol, glucose and the like can also be used to adjust the osmolality.

### Routes of Administration

The inventor has also discovered that topical administration is an effective method for delivering azithromycin and the retinoid. Instillation to the ocular surface is a localized administration method and can therefore be more effective in reaching the target area, i.e., the eye, and providing a high and localized concentration of azithromycin and the retinoid. Topical instillation avoids undesired side effects due to systemic exposure of the azithromycin and the retinoid and reduces the risk of patients from developing antibiotic resistance.

Formulations described by the present invention can be administered to the eyes of a patient by any suitable means, but are preferably administered as a liquid or gel suspension in the form of drops, spray or gel. In one embodiment, the formulation is in the form of drops, and is dropped onto the ocular surface. In another embodiment, the formulation is contained within a swab or sponge which can be applied to the ocular surface. In another embodiment, the formulation is contained within a liquid spray or ointment which can be applied to the ocular surface. In another embodiment, the formulation is injected directly into the lacrimal tissues or onto the eye surface. In a further embodiment, the formulation (e.g., in the form of drops) is first applied on a finger tip or other applicator, then applied or rubbed directly onto the lid margin. Alternatively, azithromycin and a retinoid can be applied to the eye via liposomes. Further, azithromycin and a retinoid can be infused into the tear film via a pump-catheter system. Another embodiment of the present invention involves azithromycin and a retinoid contained within a continuous or selective-release device, for example, membranes such as, but not limited to, those employed in the OCUSERT™ System (polymeric ocular inserts for the administration of drugs, Alza Corp., Palo Alto, Calif.). As an additional embodiment, azithromycin and a retinoid can be contained within, carried by, or attached to contact lenses or other compatible controlled release materials, which are placed on the eye.

In one embodiment, the azithromycin and retinoid are administered 1, 2, 3 or 4 times a day sequentially (i.e. one after the other), or co-administered together. In one embodiment, azithromycin and retinyl palmitate are admixed as one pharmaceutical composition and administered to patients by instillation on to the ocular surface. One single pharmaceutical composition and one single treatment provide ease of use and result in better compliance of patients. In another embodiment, azithromycin and retinyl palmitate are each in a separate formulation and administered separately to patients.

The daily dose to treat blepharitis can be divided among one or several unit dose administrations. The daily dose, for example, can range from one drop (about 50 μl ), one to four times a day, depending upon the age and condition of the subject. A preferred regimen is one drop of a 1% azithromycin (w/v) and 0.05% (w/v) retinyl palmitate solution, about 1 to 2 times a day. For example, a preferred dosage is one drop in each eye twice a day for two days and then once a day thereafter.

When treating blepharitis, the present method can be combined with mechanical therapy such as warm compress or lid hygiene (lid cleansing).

### Unexpected Advantages of the Present Invention

The inventor has discovered that the combined administration of azithromycin and a retinoid to a blepharitis patient has several advantages that cannot be achieved by a single administration of either azithromycin or a retinoid.

Blepharitis is a chronic disease, and subjects with blepharitis often have physical alterations to the meibomian glands and the epithelium surrounding the orifices of the meibomian glands. The inventor has discovered that the combined treatment of a retinoid and azithromycin allows for a normal meibomian gland function which improves the quality of the meibomian gland secretions, and allows for the improved secretions to be secreted and reach the ocular surface. Additionally, the anti-inflammatory properties of azithromycin reduce the side effects of irritation and inflammation during the early phase of a retinoid treatment, which enhance the tolerability of topically applied retinoid without having to reduce the amount or frequency of the retinoid application.

The invention is illustrated further by the following examples which are not to be construed as limiting the invention in scope or spirit to the specific procedures described in it.

### EXAMPLES

### Example 1

### Effect of Azithromycin and Retinyl Palmitate on Subjects with Chronic Blepharitis

### Objectives

The objective of this study is to compare the safety and efficacy of the ophthalmic formulation of a combination of azithromycin 1% (w/v) and retinyl palmitate 0.05% (w/v), versus the ophthalmic formulations of the respective active ingredients alone over a 4-week treatment period on signs and symptoms in subjects with chronic blepharitis.

### Subjects

Subjects are 18 years of age or older, and have a clinical diagnosis of moderate to severe chronic blepharitis, with a clinical sign severity score of at least 2 (moderate) on either redness or swelling (or both) of the eyelid margin and on either eyelid debris or plugging of the meibomian gland (or both). Subjects also have a symptom severity score of at least 2 (moderate) on their self-reported “most bothersome” symptom at baseline and a score of at least 2 (moderate) on any other symptom. A total of 120 subjects are enrolled in the study. Subjects do not have suspected ocular infection, lid structural abnormalities, or have presence of inflammation and/or active structural change in the iris or anterior chamber.

### Methods

This is a double-masked study. At Visit 1 (Day 1), all subjects are randomized in 1:1:1 ratio to receive either (a) combination of azithromycin and retinyl palmitate or (b) azithromycin alone or (c) retinyl palmitate alone, for 30 days. Study drug is administered as one drop in each eye BID for the first 2 days and then QD for the remainder of the study.

Study drugs are self-administered by the subjects. The subjects are prohibited in using any ocular or other medications that could confound the results of the assessments during study participation, such as antihistamines, steroids, antibiotics or preserved artificial tears.

Patients return for efficacy assessments on Visit 2 (Day 14), and Visit 3 (Day 30) as well as two follow-up visits, Visit 4 (Day 45) and Visit 5 (Day 60). Efficacy assessments performed at every visit included subject-reported symptom scores and investigator-reported scores on the signs of blepharitis.

### Scores on the Signs of Blepharitis

Investigators rate the severity of the subjects' blepharitis signs at Visits 1 through 5, according to the five classifications as listed below:

### Lid Debris (Collarettes, Clumps/Strands)

(0) Normal: clear eyelid margin
(1) Mild: occasional fragment (scurf), 1-5 collarettes
(2) Moderate: few fragments, 6-20 collarettes
(3) Severe: many fragments, 21-40 collarettes
(4) Very severe: clumps/strands, >40 collarettes

### Redness of the Eyelid Margin

(0) Normal: no redness.
(1) Mild: slightly dilated blood vessels; vessels colored pink; present in a segment of the eyelid margin.
(2) Moderate: more apparent dilation of blood vessels; vessel color more intense, whole margin of the eyelid is involved.
(3) Severe: increased vascularity of the eyelid margin, numerous and obvious dilated blood vessels, deep red in color, whole margin of the eyelid is involved.
(4) Very severe: clearly increased vascularity of the eyelid margin, large, numerous dilated blood vessels characterized by deep red color, whole margin of the eyelid is involved, noticeable conjunctival hyperemia.

### Swelling

(0) Normal: no swelling of the eyelid tissue.
(1) Mild: some swelling of the eyelid margin.
(2) Moderate: diffuse swelling of the eyelid margin.
(3) Severe: severe swelling of the eyelid margin with alterations in the eyelid folds.
(4) Very severe: swelling which clearly reduces interpalpebral aperture.

### Plugging of the Meibomian Gland (In the Middle Part of Lower Lid)

(0) Normal: clear orifices of meibomian glands in the middle part of lower lid
(1) Mild: less than ⅓ of orifices but at least one contain turbid or non-oily secretions
(2) Moderate: between ⅓ and ⅔ of orifices contain turbid or non-oily secretions
(3) Severe: more than ⅔ of orifices but not all contain turbid or non-oily secretions
(4) Very severe: All orifices plugged with turbid or non-oily secretions

### Meibomian Gland Secretion

(0) Normal: minimal clear secretion
(1) Mild: cloudy
(2) Moderate: granular
(3) Severe: paste
(4) Obstructed: no expressable secretion

### Scores on the Symptoms of Blepharitis

Subjects are asked to rate the following blepharitis symptoms at Visit 1 through 5.

### Eyelid Itching

Do your eyelids feel itchy?
(0) None: My eyelids do not feel itchy.
(1) Mild: Once in a while, my eyelids feel slightly itchy, but I do not have a desire to rub them.
(2) Moderate: Occasionally, my eyelids feel itchy, and I need to rub them.
(3) Severe: It is difficult to relieve the sensation of itchiness even when I rub my eyelids.
(4) Very severe: I have unbearable eyelid itching with an irresistible urge to rub my eyelids.

### Foreign Body Sensation/Sandiness, Grittiness

Do you feel like there's something sandy or gritty in your eye?
(0) None: My eyes do not feel sandy or gritty.
(1) Mild: I am aware of the surface of my eyes once in a while.
(2) Moderate: My eyes feel like there is something small in them occasionally.
(3) Severe: My eyes feel like there is something large or gritty in them.
(4) Very severe: I am unable to open my eyes due to feeling of a foreign body in my eyes.

### Ocular Dryness

Are your eyes feeling dry?
(0) None: My eyes do not feel dry.
(1) Mild: I am aware of dryness and have to blink to feel better.
(2) Moderate: I am aware of dryness and have to use artificial tears occasionally.
(3) Severe: I am aware of dryness and have to use artificial tears routinely.
(4) Very severe: I am aware of dryness, I always have to have artificial tears and I use them more than 6 times a day.

### Ocular Burning or Pain

Are your eyes burning or painful?
(0) None: My eyes do not burn or ache.
(1) Mild: I am aware of the surface of my eyes; they mildly burn or ache.
(2) Moderate: I feel my eyes are burning, but still tolerable.
(3) Severe: My eyes feel throbbing or fiery due to burning/pain.
(4) Very severe: I am unable to open my eyes due to burning/pain.

### Swollen/Heavy Eyelids

Do you feel like your eyelids are heavy or swollen?
(0) Normal: I don't feel that my eyelids are heavy/swollen.
(1) Mild: I feel that my eyelids are mildly heavy/swollen.
(2) Moderate: I feel my eyelids are heavy/swollen, but I can tolerate it.
(3) Severe: I feel my eyelids are heavy/swollen, I like to close my eyes for a few minutes.
(4) Very severe: I feel my eyelids are heavy/swollen and have to make an effort to keep my eyes open.
   Total Symptom Score is defined as the sum of the above five symptoms severity scores as described above.

### RESULTS

The mean scores for individual signs and symptoms and the Total Symptom Score for each group are compared for Visits 2-5 to baseline (Visit 1). A statistically significant difference (p<0.05) is observed in favor of the azithromycin and retinyl palmitate treatment group for at least one of the Visits.

### CONCLUSIONS

The above results indicate that combination of azithromycin and retinyl palmitate reduces the signs and symptoms of blepharitis significantly greater than either azithromycin or retinyl palmitate alone.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

1-12. (canceled)

13. A method for treating blepharitis, comprising the steps of:
a. identifying a patient suffering from blepharitis; and
b. topically administering to the eyes of said identified patient an effective amount of azithromycin and an effective amount of a retinoid.

14. The method according to claim 13, wherein said azithromycin and said retinoid are administered separately.

15. The method according to claim 13, wherein said azithromycin and said retinoid are co-administered in one composition.

16. The method according to claim 13, wherein said azithromycin is azithromycin monohydrate.

17. The method according to claim 13, wherein the effective amount of azithromycin is about 0.5-2% (w/v).

18. The method according to claim 13, wherein the effective amount of azithromycin is about 1% (w/v).

19. The method according to claim 13, wherein said retinoid is selected from the group consisting of retinol, retinyl palmitate, tretinoin, isotretinoin, etretinate, acitretin, tazarotene, bexarotene, and adapalene.

20. The method according to claim 19, wherein said retinoid is retinyl palmitate.

21. The method according to claim 20, wherein the effective amount of retinyl palmitate is about 0.01-1% (w/v).

22. The method according to claim 20, wherein the effective amount of retinyl palmitate is about 0.05% (w/v).

23. A method for treating blepharitis, comprising the steps of:
a. identifying a patient suffering from blepharitis; and
b. topically administering to the eyes of said identified patient a pharmaceutical composition comprising active ingredients and a pharmaceutically acceptable carrier, wherein the active ingredients comprise an effective amount of azithromycin and an effective amount of a retinoid.

24. The method according to claim 23, wherein said azithromycin is azithromycin monohydrate.

25. The method according to claim 23, wherein the effective amount of azithromycin is about 0.5-2% (w/v).

26. The method according to claim 23, wherein the effective amount of azithromycin is about 1% (w/v).

27. The method according to claim 23, wherein said retinoid is selected from the group consisting of retinol, retinyl palmitate, tretinoin, isotretinoin, etretinate, acitretin, tazarotene, bexarotene, and adapalene.

28. The method according to claim 27, wherein said retinoid is retinyl palmitate.

29. The method according to claim 28, wherein the effective amount of retinyl palmitate is about 0.01-1% (w/v).

30. The method according to claim 28, wherein the effective amount of retinyl palmitate is about 0.05% (w/v).

31. A pharmaceutical composition for treating blepharitis comprising active ingredients and a pharmaceutically acceptable carrier, wherein the active ingredients consist essentially of 0.5-2% (w/v) of azithromycin and 0.01-1% (w/v) retinyl palmitate.
